(19) United States
(12) Patent Application Publication
(10) Pub. No.: US 2018/0249049 A1
(43) Pub. Date: Aug. 30, 2018

(54) ENDOSCOPE

(71) Applicant: OLYMPUS CORPORATION, Tokyo (JP)

(72) Inventor: Kazuya MAEDA, Kamiina-gun (JP)

(73) Assignee: OLYMPUS CORPORATION, Tokyo (JP)

(21) Appl. No.: 15/969,898

(22) Filed: May 3, 2018

Related U.S. Application Data
(63) Continuation of application No. PCT/JP2015/081444, filed on Nov. 9, 2015.

(51) Int. Cl. H04N 5/225

(52) U.S. Cl. CPC H04N 5/2253; A61B 1/00124; H04N 5/2257

(57) ABSTRACT

An endoscope includes: a distal end rigid portion in which an image pickup device is disposed; a bending portion for changing a direction of the distal end rigid portion; and a flexible portion extended from the bending portion, and further includes a flexible wiring board including deformation portions that deform due to bending deformation of the bending portion and non-deformation portions that do not deform due to the bending deformation of the bending portion, the deformation portions and non-deformation portions being alternately provided in a continuous manner, the wiring board including a plurality of wirings connected with the image pickup device.

### CROSS REFERENCE TO RELATED APPLICATION

This application is a continuation application of PCT/JP2015/081444 filed on Nov. 9, 2015, the entire contents of which are incorporated herein by this reference.

### BACKGROUND OF INVENTION

### 1. Field of the Invention

The present invention relates to an endoscope including a distal end rigid portion in which an image pickup device is disposed, a bending portion for changing a direction of the distal end rigid portion, and a flexible portion extended from the bending portion.

### 2. Description of the Related Art

For the purpose of improving operability of an endoscope and achieving low invasion, reduction of the size and length of a distal end rigid portion in which an image pickup device is disposed is an essential problem to be solved. Also a wiring board on which electronic components for driving the image pickup device and processing image pickup signals are mounted is also disposed in the distal end rigid portion. A signal cable for transmitting a drive signal and an image pickup signal to a processor is mounted to a bonding electrode of the wiring board.

Japanese Patent Application Laid-Open Publication Nos. 62-199057 and 62-199058 disclose endoscopes each including an elongated belt-like (strip-like) wiring board. The belt-like wiring board is deformable, since the wiring board is bent in a bellows-like shape or wound in a spiral shape. That is, the wiring board of each the endoscopes is used instead of a signal cable.

Note that the bonding electrode to which electronic components are mounted is not disposed on the wiring board of each of the endoscopes. Therefore, the endoscopes have not contributed to the reduction of the length and size of the distal end rigid portion.

### SUMMARY OF THE INVENTION

An endoscope according to an embodiment of the present invention includes: a distal end rigid portion in which an image pickup device is disposed; a bending portion for changing a direction of the distal end rigid portion; a flexible portion extended from the bending portion; and a belt-like flexible wiring board including a plurality of wirings connected with the image pickup device, and the wiring board arranged inside the bending portion includes a plurality of deformation portions that deform due to bending deformation of the bending portion and a plurality of non-deformation portions that do not deform due to the bending deformation of the bending portion, the plurality of deformation portions and the plurality of non-deformation portions being alternately provided in a continuous manner.

### BRIEF DESCRIPTION OF THE DRAWINGS

FIG. 1 is a perspective view of an endoscope according to a first embodiment.

FIG. 2 is a side view of an image pickup module of the endoscope according to the first embodiment.

FIG. 3 is a development view of a wiring board of the image pickup module of the endoscope according to the first embodiment.

FIG. 4 is a partial perspective view of the wiring board of the image pickup module of the endoscope according to the first embodiment.

FIG. 5 is a partial perspective view of a wiring board of an image pickup module of an endoscope according to a first modified example of the first embodiment.

FIG. 6 is a development view of a wiring board of an image pickup module of an endoscope according to a second embodiment.

FIG. 7 is a partial perspective view of a wiring board of the image pickup module of the endoscope according to the second embodiment.

FIG. 8 is a development view of a wiring board of an image pickup module of an endoscope according to a third embodiment.

FIG. 9 is a perspective view of the wiring board of the image pickup module of the endoscope according to the third embodiment.

FIG. 10 is a side view illustrating a deformation of the wiring board of the image pickup module of the endoscope according to the third embodiment.

FIG. 11 is a top view illustrating a deformation of the wiring board of the image pickup module of the endoscope according to the third embodiment.

### DETAILED DESCRIPTION OF THE PREFERRED EMBODIMENT(S)

### First Embodiment

First, description will be made on an endoscope 1 according to the present embodiment.

Note that, in the description below, each of the drawings based on each embodiment is a pattern diagram, and care should be taken to the fact that the relationship between the thicknesses and widths of the respective parts, a ratio of the thickness of a certain part to that of another part, and the like are different from the actual ones, and there is a case where the respective drawings include parts in which the relationships and ratios among the dimensions are different. In addition, there is a case where illustration of some of the constituent elements will be omitted.

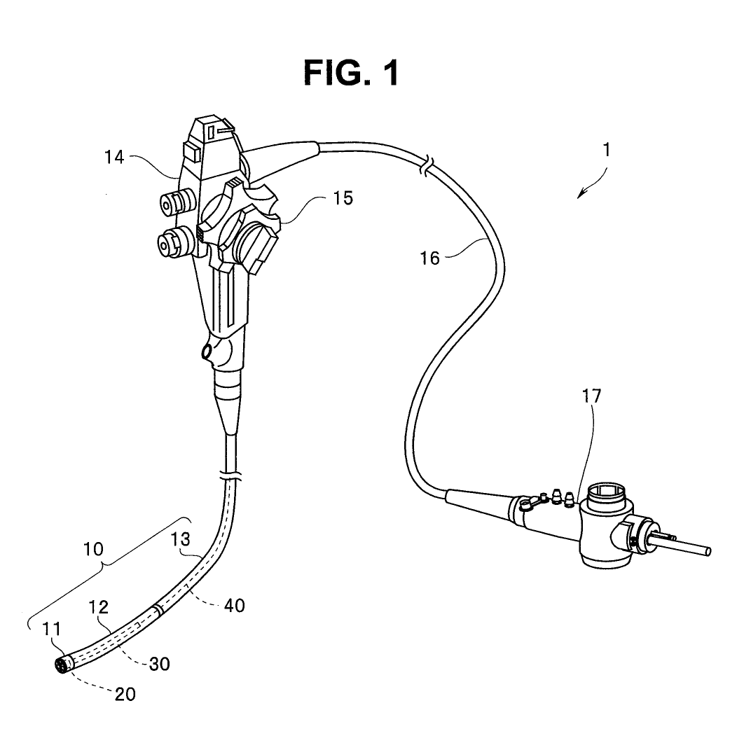

As shown in FIG. 1, an endoscope 1 includes an elongated insertion portion 10, an operation portion 14, a universal cord 16, and a connector 17. The insertion portion 10 includes a distal end rigid portion 11, a bending portion 12 for changing the direction of the distal end rigid portion 11, and an elongated flexible portion 13 extended from the bending portion 12. An image pickup device 20 is disposed in the distal end rigid portion 11. A connector 17 disposed at a proximal end portion of the universal cord 16 is connected with a processor (not shown).

An angle knob 15 that operates the bending portion 12 is disposed at the operation portion 14. For example, when the angle knob 15 mechanically connected to the bending portion 12 through an operation wire (not shown) is rotated, the bending portion 12 is bent to be deformed in four directions, i.e., up, down, left, and right directions. In accordance with the bending deformation of the bending portion 12, the direction of the distal end surface of the distal end rigid portion 11 extended from the distal end of the bending portion 12 is changed, that is, the image pickup field of view direction of the image pickup device 20 is changed.

The image pickup device 20 is connected with a plurality of signal cables 40 for transmitting/receiving signals through wirings 35 (see FIG. 3) of the wiring board 30. In the endoscope 1, a rear end portion of the wiring board 30 is disposed in the bending portion 12. In other words, the part of the wiring board 30, which is arranged in the distal end rigid portion 11, is referred to as a front end portion, and the other part of the wiring board 30, which is arranged in the bending portion 12, is referred to as the rear end portion.

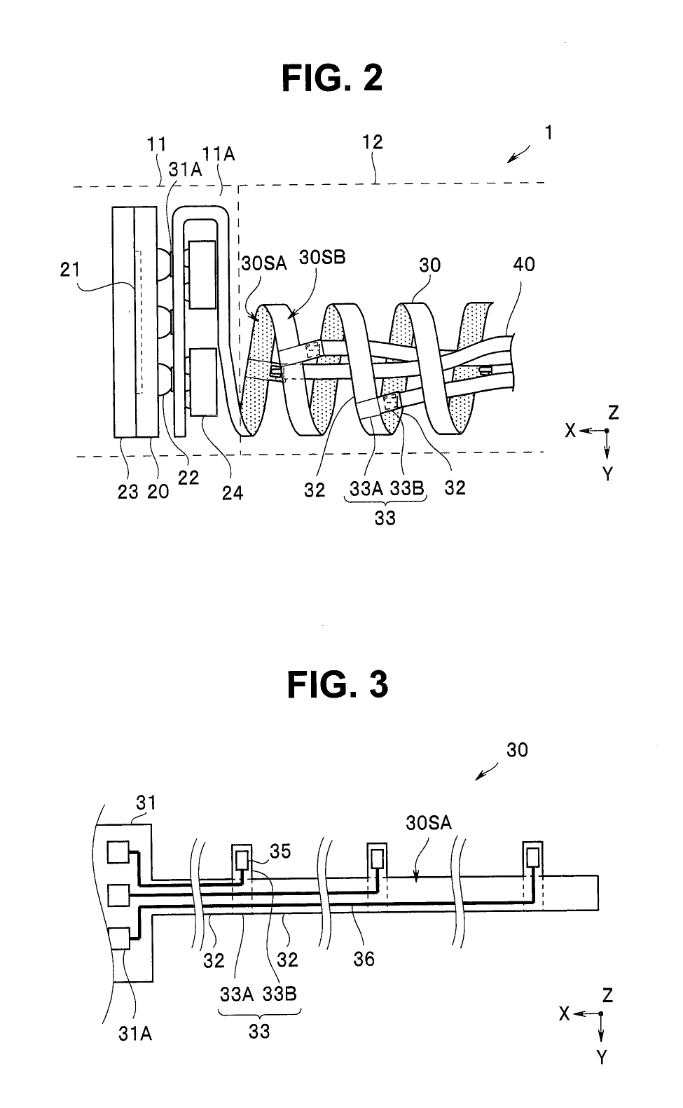

That is, as shown in FIG. 2, the image pickup device 20 and the front end portion of the elongated wiring board 30 are disposed in the distal end rigid portion 11. An image pickup optical system (not shown) including a cover glass 23 is disposed on a light-receiving surface of the image pickup device 20, a light-receiving portion 21 being formed on the light-receiving surface. Electrodes including a plurality of external connecting electrodes 22 are disposed on a rear surface opposed to the light-receiving surface of the image pickup device 20.

Note that, the direction in which the distal end rigid portion 11 of the elongated insertion portion 10 is disposed is referred to as “front side”. In FIG. 2, etc., the “front side” is a direction in which a value of an X-axis increases.

The plurality of external connecting electrodes 22 of the image pickup device 20 are bonded to a front end portion 31 of the wiring board 30, and also electronic components 24 such as chip capacitors are mounted to the front end portion 31. The distal end rigid portion 11 is sealed by a resin 11A, for example. That is, the front end portion 31 of the wiring board 30 is fixed to the inside of the distal end rigid portion 11 by the sealing resin 11A.

The wiring board 30 is a flat belt-like shape in which the length is longer than the width. A flexible base of the wiring board 30 is made of polyimide of 3 mm in width and 5 cm in length, for example. The wiring board 30 is preferably made of a flexible resin having thickness from 1 μm or more to 200 μm or less, and more preferably having a thickness from 10 μm or more to 50 μm or less so that deformation portions 32 easily deform, as described later.

The wiring board 30 includes a plurality of wirings 36 each of which is made of a conductor such as copper connected with the image pickup device 20. When the wiring board 30 is in a flat state, the flexible wiring board 30 easily deforms in a direction (Z-axis direction) orthogonal to the plane (XY plane) of the wiring board 30, but does not deform in a direction (X-axis direction/Y-axis direction) parallel to the plane (XY plane) of the wiring board 30.

In contrast, as shown in FIG. 2, the belt-like wiring board 30 is wound in a spiral shape in the endoscope 1. Therefore, the wiring board 30 easily deforms also in up, down, left and right directions (Y-axis direction, Z-axis direction). That is, the rear end portion of the wiring board 30, which is disposed inside the bending portion 12, easily deforms in accordance with the deformation of the bending portion 12.

The wound wiring board 30 easily deforms in up, down, left, and right directions. However, the wiring board 30 is not configured to deform over the entire length thereof. That is, the wiring board 30 includes a plurality of deformation portions 32 that easily deform due to the bending deformation of the bending portion 12, and a plurality of non-deformation portions 33 that do not deform as easily as the deformation portions 32, the deformation portions 32 and the non-deformation portions 33 being alternately provided in a continuous manner. Note that the plurality of non-deformation portions 33 are arranged in FIG. 2. However, the plurality of non-deformation portions 33 do not have to be arranged at equal intervals. For example, two non-deformation portions 33 may be arranged side by side with an extremely short deformation portion 32 being sandwiched therebetween. Arranging the non-deformation portions 33 and the deformation portions 32 continuously in such a manner means that the plurality of non-deformation portions and the plurality of deformation portions are alternately provided in a continuous manner.

Each of the non-deformation portions 33 of the belt-like wiring board 30 includes a rectangular-shaped connecting portion 33A, both side surfaces of which are respectively continuous with the deformation portions 32 located anterior to and posterior to the connecting portion 33A, and an extending portion 33B extended from the side surface orthogonal to the side surfaces that are continuous with the deformation portions 32. A different looking at such a configuration is that each of the non-deformation portions 33 deforms less than the deformation portions 32, since each of the non-deformation portions 33 includes the extending portion 33B. In addition, the extending portion 33B deforms far less than the connecting portion 33A.

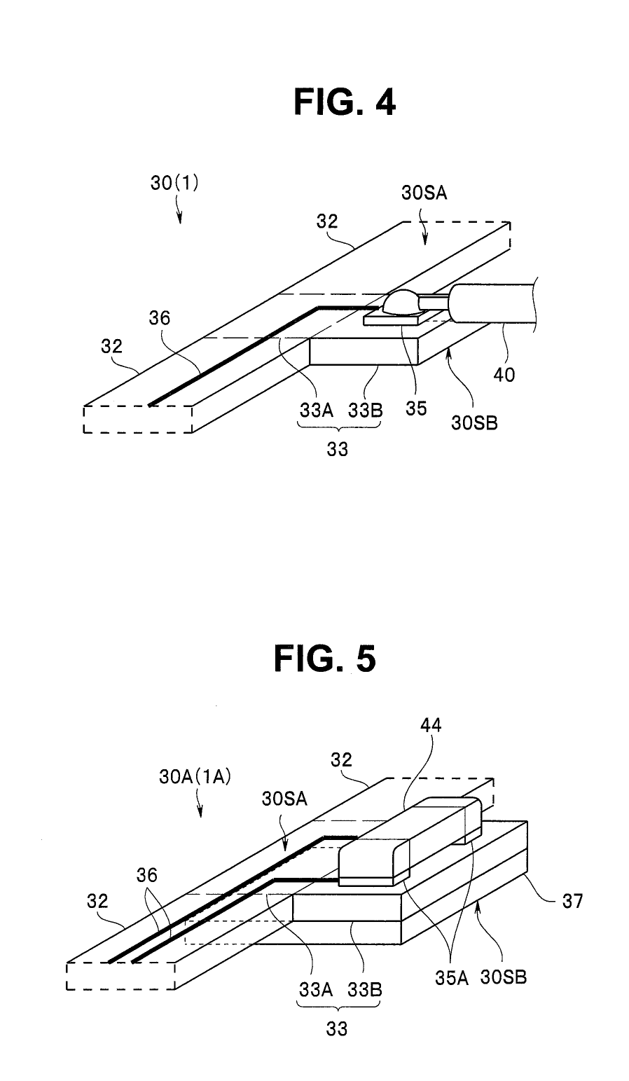

Bonding electrodes 35, each of which is made of a conductive film such as copper and electrically connected with the image pickup device 20 through each of the wirings 36, are disposed on the respective extending portions 33B. As shown in FIG. 4, in the endoscope 1, the bonding electrodes 35 are bonded respectively with signal cables 40, which are other members, by soldering, for example.

In the endoscope 1, the extending portions 33B of the wiring board 30, on which the bonding electrodes 35 are disposed, are not arranged in the distal end rigid portion 11 but in the bending portion 12. Therefore, the length from the distal end surface of the cover glass 23 to the front end portion 31 of the wiring board 30 is 1.2 mm to 2.0 mm, for example.

In the endoscope 1, the bonding portions between the wiring board 30 and the respective signal cables 40 are arranged inside the bending portion 12, which results in the short length and small size of distal end rigid portion 11, for example, the length of the distal end rigid portion 11 is equal to or shorter than 5 mm.

Note that some of the bonding electrodes 35 may be arranged inside the distal end rigid portion 11, and the bonding portions may be fixed with the sealing resin 11A.

If the base of the wiring board deforms, stress is applied to the bonding portions between the bonding electrodes 35 and the signal cables 40, which is likely to cause a degradation of bonding reliability.

In the endoscope 1, the deformation portions 32 of the wiring board 30, which are arranged in the bending portion 12, deform due to the bending deformation of the bending portion 12. On the other hand, though the extending portions 33B are arranged inside the bending portion 12, the extending portions 33B hardly deform even if the bending portion 12 is bent to be deformed. Therefore, the bonding reliability between the bonding electrodes 35 and the signal cables 40 is high.

Note that, in the endoscope 1, the wirings 36 and the bonding electrodes 35 are disposed only on a top surface 30SA of the wiring board 30, but may be disposed also on a rear surface 30SB of the wiring board 30. In addition, the wiring board 30 may include a through wiring for connecting the top surface 30SA and the rear surface 30SB, or may be a multilayer wiring board. Furthermore, the extending portions 33B do not have to be extended from all of the connecting portions 33A. In addition, the bonding electrodes 35 do not have to be disposed on all of the plurality of extending portions 33B. Furthermore, the signal cables 40 do not have to be bonded to all of the bonding electrodes 35. The extending portions may be extended respectively from the both side surfaces of one connecting portion 33A.

Note that the plurality of deformation portions 32 may be different in the length in the longitudinal direction, and the like. In addition, the plurality of non-deformation portions 33 may be different in the length in the longitudinal direction, the extending direction and the shape of the extending portion 33B, the number of the bonding electrodes 35 arranged on one extending portion 33B, and the like. In addition, the bonding portions between the bonding electrodes 35 and the signal cables 40 may be sealed with resin.

The wiring board 30 is inserted through the front side of the bending portion 12, and the signal cables 40 are inserted through the rear side of the bending portion 12. However, the endoscope according to the present embodiment may include a long wiring board which is inserted through the bending portion 12 over the entire length thereof and which has a rear end arranged inside the flexible portion 13. However, it is not preferable to provide a long wiring board which is inserted through the flexible portion 13 over the entire length thereof, since providing such a long wiring board will lead to the degradation of reliability.

### Modified Example of First Embodiment

Next, description will be made on an endoscope 1A according to a modified example of the first embodiment. Since the endoscope 1A is similar to the endoscope 1, the same constituent elements are attached with the same reference numerals and descriptions thereof will be omitted.

As shown in FIG. 5, in the endoscope 1A, the electronic components 44 such as chip capacitors are surface-mounted on a plurality of bonding electrodes 35A respectively disposed on extending portions 33B of a wiring board 30A.

In the endoscope 1A, the rear end portion of the wiring board 30A on which the electronic components 44 are surface-mounted is arranged inside the bending portion 12. Therefore, the length of the distal end rigid portion 11 of the endoscope 1A is shorter and smaller than that of the endoscope 1. In addition, in the endoscope 1A, even if the bending portion 12 deforms, the extending portions 33B hardly deform. Therefore, the bonding reliability between the bonding electrodes 35A and the electronic components 44 is secured.

Note that, as shown in FIG. 5, a reinforcing member 37 is pasted on each of the non-deformation portions 33 of the endoscope 1A. Therefore, the thickness of each of the non-deformation portions 33 is larger than that of each of the deformation portions 32.

The reinforcing member 37 is not an essential constituent element. However, the non-deformation portions 33 having a thickness larger than that of the deformation portions 32 are harder to deform and the bonding reliability is secured more surely. The reinforcing member 37 is made of a polyimide film, a non-flexible resin board, or the like, having the same thickness as that of the flexible base of the wiring board 30A, for example.

Note that the reinforcing member 37 may be pasted only on each of the extending portions 33B of the non-deformation portions 33. In addition, needless to say, it is preferable that the thickness of each of the non-deformation portions 33 is larger than that of each of the deformation portions 32 also in the wiring board 30 of the endoscope 1 according to the first embodiment that has been already described above.

In the endoscopes 1 and 1A, the bonding electrodes 35 and 35A on which other members are mounted are respectively disposed on the non-deformation portions 33. In the endoscope 1, the other members are the signal cables 40, and in the endoscope 1A, the other members are the electronic components 44. It is needless to say that the endoscope according to the embodiment may include the signal cables 40 and the electronic components 44 as other members, as described later.

As described above, the endoscope 1 includes the image pickup device 20, the wiring board 30, the signal cables 40 and/or the electronic components 44.

The image pickup device 20 photoelectrically converts the object image entered from the front to generate an image pickup signal, and includes, on the rear surface of the light-receiving surface, a plurality of electrodes including the external connecting electrodes 22 that output the image pickup signal. The flexible wiring board 30 includes the wirings 36 which are connected with the plurality of electrodes of the image pickup device 20 and which guide the image pickup signal rearward. The signal cables 40 are respectively connected with the bonding electrodes 35 arranged on the wiring board 30, to guide the image pickup signal more rearward. The electronic components 44 are mounted to the bonding electrodes 35A arranged on the wiring board 30.

The elongated belt-like wiring board 30 is configured such that the plurality of deformation portions 32 as the flexible portions and the plurality of non-deformation portions 33 as the non-flexible portions are alternately arranged in the longitudinal direction, and the plurality of bonding electrodes 35 and 35A are arranged only on the non-deformation portions 33 and are not arranged at least on the deformation portions 32 that are arranged inside the bending portion.

Note that, as in the endoscope 1, the electronic components 24 may be mounted on the front end portion of the wiring board 30 which is arranged in the distal end rigid portion 11. However, in the endoscope including all the electronic components inside the bending portion 12, the distal end rigid portion 11 is shorter and smaller.

In addition, the sealing resin covering the bonding portions between the bonding electrodes 35 and the signal cables 40 may have a function as the reinforcing member 37. Furthermore, the sealing resin may cover not only the bonding portions but also the entire surface of each of the extending portions 33B, and may further cover the entire surface of each of the non-deformation portions 33, that is, up to the surface of each of the connecting portions 33A.

### Second Embodiment

Next, description will be made on an endoscope 1B according to the second embodiment. Since the endoscope 1B is similar to the endoscopes 1 and 1A, the same constituent elements are attached with the same reference numerals and descriptions thereof will be omitted.

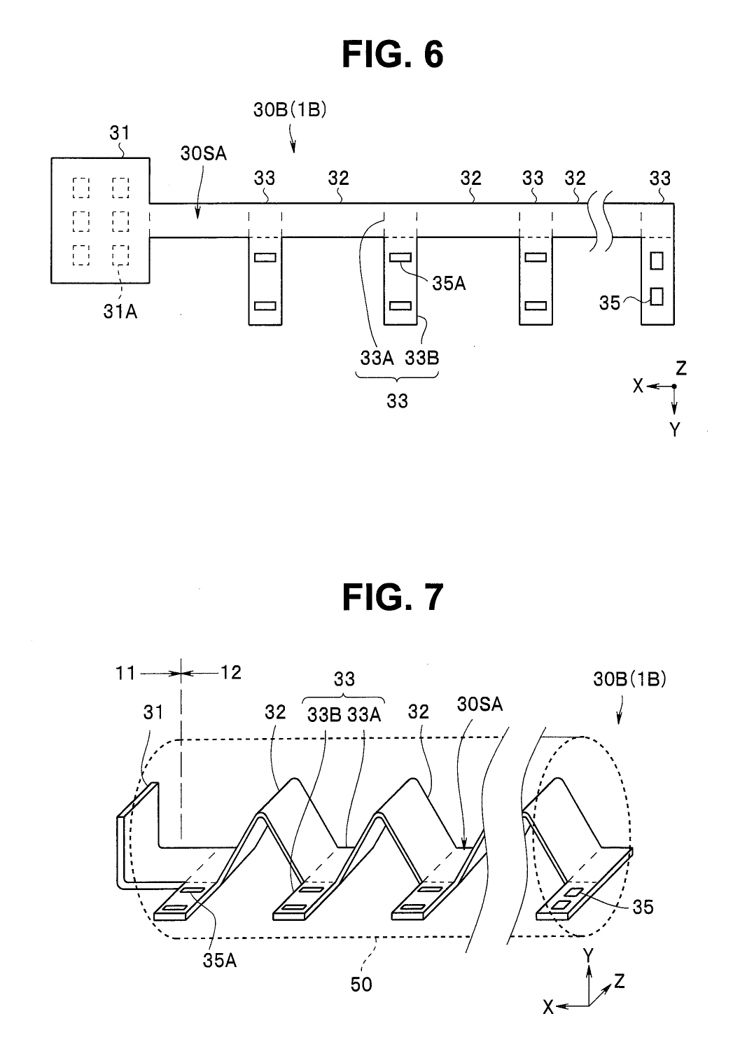

As shown in FIGS. 6 and 7, similarly as in the wiring boards 30 and 30A of the endoscopes 1 and 1A, a wiring board 30B of the endoscope 1B is configured such that a plurality of deformation portions 32 that deform due to the bending deformation of the bending portion 12 and a plurality of non-deformation portions 33 that do not deform due to the bending deformation of the bending portion 12 are alternately provided in a continuous manner, and the wiring board 30B includes wirings connected with the image pickup device 20.

As shown in FIG. 7, the belt-like wiring board 30B is configured such that the plurality of deformation portions 32 are bent in a V-shape in the direction (Z-axis direction) orthogonal to the longitudinal direction (X direction) and formed in a bellows-like shape.

The bonding electrodes 35A on which the electronic components (not shown) are respectively mounted or the bonding electrodes 35 to which the signal cables (not shown) are respectively bonded are disposed on the extending portions 33B of the wiring board 30B. Note that only the signal cables may be bonded to the wiring board 30B. Furthermore, all the electronic components may be arranged inside the bending portion 12.

The wiring board 30B easily deforms not only in the longitudinal vertical direction (Z-axis direction) but also in the in-plane direction (X-axis direction and Y-axis direction), since the deformation portions 32 have a bellows-like shape formed by each of the deformation portions 32 being bent in a V-shape.

Similarly as the wiring board 30 of the endoscope 1, the wiring board 30B of the endoscope 1B, except for the front end portion thereof bonded to the image pickup device, is disposed inside the bending portion 12. However, even if the bending portion 12 is bent to be deformed, the deformation portions 32 easily deform in accordance with the deformation of the bending portion. Therefore, the non-deformation portions 33, specifically the extending portions 33B hardly deform. Such a configuration ensures the bonding reliability of the signal cables and the electronic components.

That is, the belt-like wiring board 30B includes the deformation portions 32 folded in a V-shape at a plurality of folds in a direction orthogonal to the longitudinal direction, and further includes the non-deformation portions 33 each of which includes an extending portion 33B extended from the side surface thereof. The defolination portions 32 are formed in the bellows-like shape, but the non-deformation portions 33 including the extending portions 33B on which the bonding electrodes 35A are respectively disposed maintain a flat state. The wiring board 30B is capable of being flexed not only in the up/down direction (Z-axis direction) but also in the left/right direction (Y-axis direction) by the deformation portions 32 having the bellows-like shape. On the other hand, the deformation of the non-deformation portions 33 is restrictive.

Therefore, in the endoscope 1B, the distal end rigid portion 11 is short and small, and the bonding reliability of the signal cables 40 and the electronic components 44 is high.

Note that the wiring board 30B is preferably inserted into a bendable cylindrical-shaped exterior member 50, as shown in FIG. 7. The exterior member 50 may be a resin tube or a meshed metal cylinder, as long as the exterior member is deformable in up, down, left and right directions.

Inside the bending portion 12, an optical fiber that guides the illumination light is also inserted, for example. Even if the wiring board 30B inserted in the exterior member 50 deforms due to the bending of the bending portion 12, there is no possibility that the wiring board 30B affects the optical fiber and the like inserted through the inside of the bending portion 12.

Note that, needless to say, it is preferable that the thickness of each of the non-deformation portions 33 is larger than that of each of the deformation portions 32 also in the endoscope 1B.

### Third Embodiment

Next, description will be made on an endoscope 1C according to the third embodiment. Since the endoscope 1C is similar to the endoscope 1 and the like, the same constituent elements are attached with the same reference numerals and descriptions thereof will be omitted.

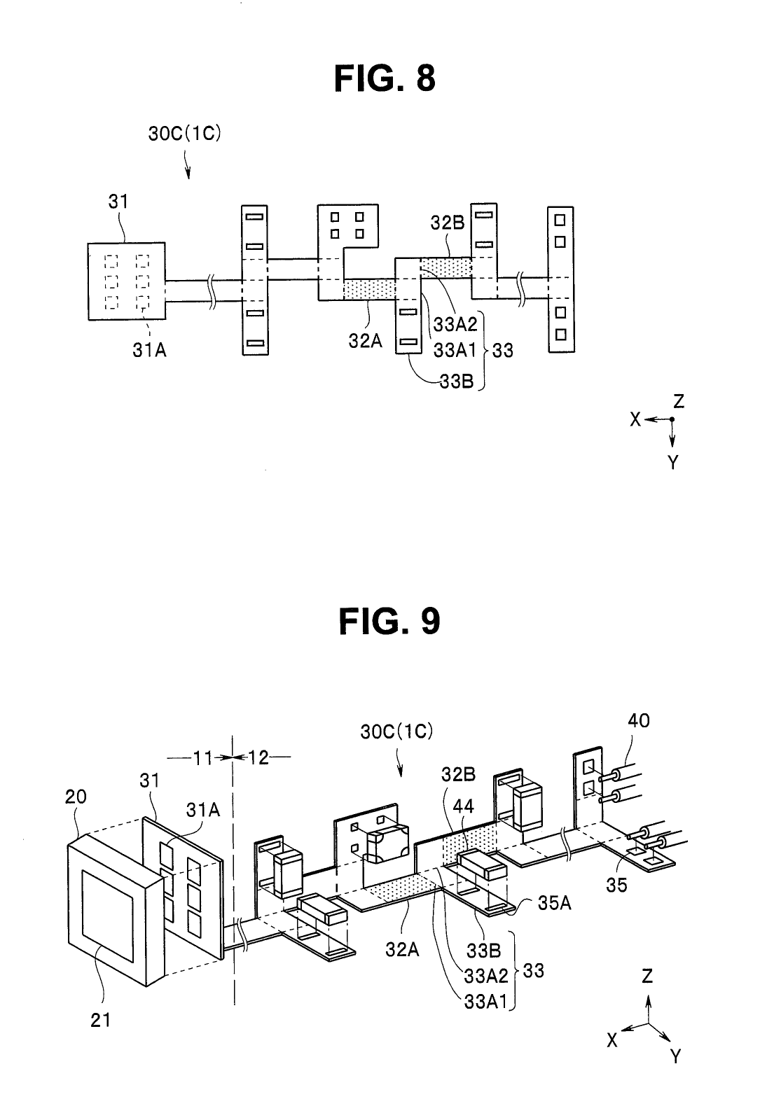

As shown in FIGS. 8 and 9, similarly as the wiring board 30 of the endoscope 1, a belt-like wiring board 30C of the endoscope 1C is configured such that a plurality of deformation portions 32 that deform due to the bending deformation of the bending portion 12 and a plurality of non-deformation portions 33 that do not deform due to the bending deformation of the bending portion 12 are alternately provided in a continuous manner, and the wiring board 30C includes a plurality of wirings connected with the image pickup device 20.

As shown in FIG. 8, the belt-like wiring board 30C of the endoscope 1C is configured such that a connecting portion 33A of each of the non-deformation portions 33 includes a rectangular-shaped first connecting portion 33A1 and a rectangular-shaped second connecting portion 33A2.

As shown in FIG. 9, the second connecting portion 33A2 is bent so as to be orthogonal to the first connecting portion 33A1. Therefore, the first connecting portion 33A1 is located on the XY plane, while the second connecting portion 33A2 is located on the XZ plane.

A rectangular-shaped first deformation portion 32A located anterior to each of the connecting portions 33A is connected with the forward side surface of each of the first connecting portions 33A1. On the other hand, a rectangular-shaped second deformation portion 32B located posterior to each of the connecting portions 33A is connected with the rearward side surface of each of the second connecting portions 33A2. Therefore, the first deformation portions 32A are located on the XY plane, while the second deformation portions 32B are located on the XZ plane.

Each of the connecting portions 33A further includes an extending portion 33B extended from the side surface thereof, and the bonding electrodes 35 and 35A connected with the wirings are disposed on the extending portion 33B. The signal cables 40 are respectively bonded to the bonding electrodes 35, and the electronic components 44 are mounted to the bonding electrodes 35A.

As described above, the elongated wiring board 30C is folded at the folds parallel to the longitudinal direction (optical axis direction: X direction). That is, the wiring board 30C is arranged on the XY plane and the XZ plane that form an angle of 90 degrees. That is, the first deformation portions 32A or the second deformation portions 32B are arranged either on the XY plane or the XZ plane in the direction orthogonal to the optical axis direction, and the non-deformation portions 33 are arranged both on the XY plane and XZ plane. The bonding electrodes 35, 35A are arranged on the extending portions 33B extended respectively from the non-deformation portions 33.

The plurality of extending portions 33B are different in the number and the arrangement of the bonding electrodes 35A, depending on the electronic components mounted to the respective extending portions 33B. For example, two bonding electrodes 35A are disposed on the extending portion 33B on which a two-terminal electronic component such as a chip capacitor is mounted. On the other hand, four bonding electrodes 35A are disposed on the extending portion 33B on which a four-terminal electronic component is mounted.

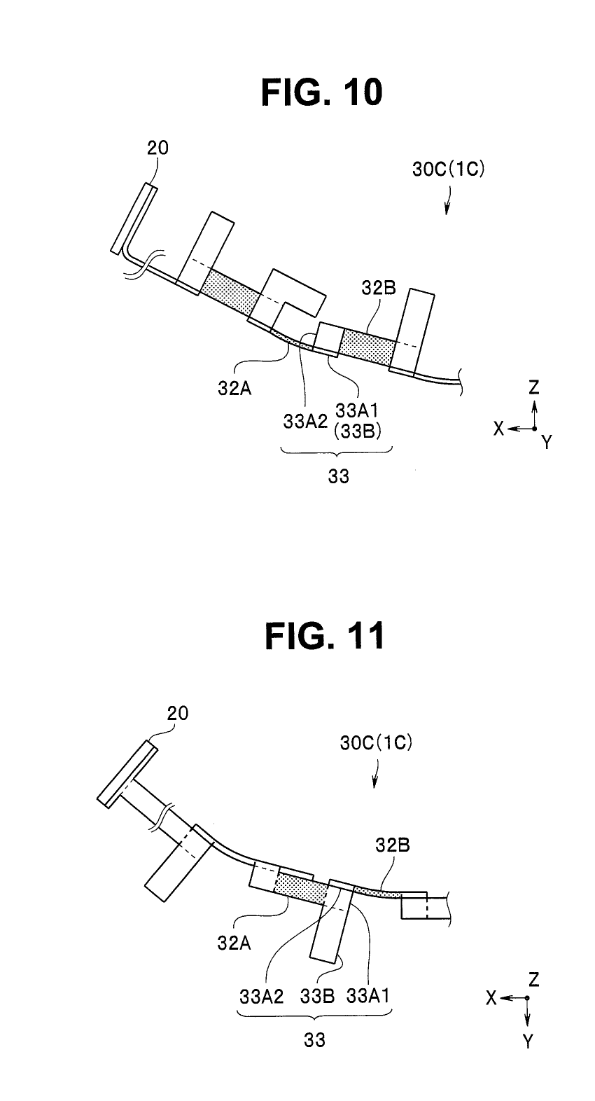

FIG. 10 is a side view illustrating a deformation of the wiring board 30C due to the deformation of the bending portion 12 in the up/down direction (Z-axis direction). When the bending portion 12 is bent in the up direction (Z-axis value increasing direction), the first deformation portions 32A located on the XY plane deform. The second deformation portions 32B and the non-deformation portions 33 do not deform. The same is true on the case where the bending portion 12 is bent in the down direction (Z-axis value decreasing direction).

On the other hand, FIG. 11 is a top view illustrating the deformation of the wiring board 30C due to the deformation of the bending portion 12 in the left/right direction (Y-axis direction). When the bending portion 12 is bent in the right direction (Y-axis value decreasing direction), the second deformation portions 32B located on the XZ plane deform. The first deformation portions 32A and the non-deformation portions 33 do not deform. The same is true on the case where the bending portion 12 is bent in the left direction (Y-axis value increasing direction).

Similarly as in the wiring board 30 of the endoscope 1, the wiring board 30C of the endoscope 1C, except for the front end portion 31 bonded to the image pickup device 20, is disposed inside the bending portion 12. Therefore, the distal end rigid portion 11 is short and small. Even if the bending portion 12 is bent to be deformed, the deformation portions 32A and 32B easily deform in accordance with the deformation of the bending portion. Therefore, the non-deformation portions 33, specifically the extending portions 33B hardly deform. Such a configuration ensures the bonding reliability of the signal cables 40 and the electronic components 44.

Furthermore, the wiring board 30C of the endoscope 1C can be easily manufactured than the wiring board 30, and the like.

Note that, needless to say, it is preferable that the thickness of each of the non-deformation portions 33 is larger than that of each of the deformation portions 32 also in the endoscope 1C.

The present invention is not limited to the above-described embodiments and the modified example, etc., and various changes, combination, and application are possible without departing from the gist of the invention.

What is claimed is:

1. An endoscope comprising:
a distal end rigid portion in which an image pickup device is disposed;
a bending portion for changing a direction of the distal end rigid portion;
a flexible portion extended from the bending portion; and
a belt-like flexible wiring board including a plurality of wirings connected with the image pickup device,
wherein the wiring board arranged inside the bending portion includes a plurality of deformation portions that deform due to bending deformation of the bending portion and a plurality of non-deformation portions that do not deform due to the bending deformation of the bending portion, the plurality of deformation portions and the plurality of non-deformation portions being alternately provided in a continuous manner,
a bonding electrode to which another member is mounded is disposed on at least one of the plurality of non-deformation portions,
each of the plurality of non-deformation portions includes an extending portion extended from a side surface of the each of the non-deformation portions, and
the bonding electrode is disposed on the extending portion.

2. The endoscope according to claim 1, further comprising a signal cable, wherein the signal cable is mounted to the bonding electrode, as the other member.

3. The endoscope according to claim 1, further comprising an electronic component, wherein the electronic component is mounted to the bonding electrode, as the other member.

4. The endoscope according to claim 1, wherein each of the plurality of non-deformation portions has a thickness larger than a thickness of each of the plurality of deformation portions.

5. The endoscope according to claim 1, wherein the wiring board is inserted inside a cylindrical-shaped exterior member that is configured to be bent and deformed.

6. The endoscope according to claim 1, wherein the wiring board is wound in a spiral shape.

7. The endoscope according to claim 1, wherein each of the deformation portions of the wiring board is folded in a V-shape.

8. The endoscope according to claim 1, wherein
a connecting portion of each of the non-deformation portions, which is connected with the deformation portions located anterior to and posterior to each of the non-deformation portions, includes a first connecting portion, and a second connecting portion bent so as to be orthogonal to the first connecting portion,
a first deformation portion provided anterior to the connecting portion so as to be continuous with the connecting portion is connected with a forward side surface of the first connecting portion,
a second deformation portion provided posterior to the connecting portion so as to be continuous with the connecting portion is connected with a rearward side surface of the second connecting portion, and
the connecting portion further includes the extending portion extended from a side surface of the connecting portion.
